(19) United States
(12) Patent Application Publication
(10) Pub. No.: US 2020/0289525 A1
(43) Pub. Date: Sep. 17, 2020

(54) Methods of Increasing Microbial Diversity of a Skin Microbiota

(71) Applicant: The Procter & Gamble Company, Cincinnati, OH (US)

(72) Inventors: Karl Shiqing WEI, Mason, OH (US); He ZHAO, Singapore (SG); Jian XU, Qingdao (CN)

(21) Appl. No.: 16/352,413

(22) Filed: Mar. 13, 2019

(51) Int. Cl. A61K 31/555; A61K 31/01; A61K 31/23; A61K 8/58; A61K 8/46; A61K 31/315; A61Q 19/10; A61P 17/00

(52) U.S. Cl. CPC A61K 31/555; A61K 31/01; A61K 31/23; A61P 17/00; A61K 8/463; A61K 31/315; A61Q 19/10; A61K 8/58

(57) ABSTRACT

Materials and methods for selectively increasing the diversity of the skin microbiota of a subject with an amenable skin condition are provided in disclosing methods for using compositions comprising a zinc compound, a biocompatible surfactant, and a lipid. Amenable skin conditions include healthy skin and skin exhibiting atopic dermatitis (with or without lesions), skin dysbiosis and/or acne.

### FIELD

The disclosure relates to the fields of cosmetic and therapeutic treatments of mammalian skin.

### BACKGROUND

Trillions of bacteria, fungus, viruses, archaea and small arthropods colonize the skin surface, collectively comprising the skin microbiota. The skin microbiota can be impacted by things such as the environment, illness, infections, and materials placed in contact with the skin. While people generally think of bacteria on the skin as a bad thing, there are bacteria on the skin which are beneficial. Skin conditions, such as atopic dermatitis, can interrupt the balance of the skin microbiome allowing bad bacteria to flourish, while the good bacteria are repressed or are unable to appropriately populate the skin. This results in a decreased diversity of the skin microbiota. In addition, compositions applied to the skin can exacerbate an unbalanced skin microbiota or even cause an unbalance. As such, there is a need for compositions which can increase the diversity of a skin microbiota.

### SUMMARY

Included herein are methods of selectively increasing microbial diversity of the skin microbiota of amenable skin comprising selecting a rinse-off multi-phase skin improvement composition comprising a cleansing phase comprising an anti-microbial compound, and a lathering biocompatible surfactant; and a benefit phase comprising a benefit agent comprising a lipid; and administering an effective amount of the multi-phase skin improvement composition, thereby selectively increasing the microbial diversity of the skin microbiota compared to the microbial diversity of the skin microbiota prior to administration of the multi-phase skin improvement composition.

Also included herein are methods for selectively increasing the diversity of skin microbiota of a subject with an amenable skin condition, comprising selecting a rinse-off multi-phase skin improvement composition which selectively targets Staphylococcus, wherein the multi-phase skin improvement composition comprises a cleansing phase comprising a surfactant and zinc pyrithione; and a benefit phase comprising a benefit agent.

Other features and advantages of the present disclosure will become apparent from the following detailed description, including the drawings. It should be understood, however, that the detailed description and the specific examples are provided for illustration only, because various changes and modifications within the spirit and scope of the invention will become apparent to those skilled in the art from the detailed description.

### BRIEF DESCRIPTION OF THE DRAWINGS

FIG. 1 is a set of histograms showing the identities and relative abundances of 14 bacterial genera and one catch-all category on the skin of healthy subjects, subjects with skin exhibiting atopic dermatitis with lesions, and subject with skin exhibiting atopic dermatitis without lesions (non-lesions). Staphylococcus is the predominant bacterial genus associated with atopic dermatitis. The histograms show the relationship between atopic dermatitis and microbiota diversity;

FIG. 2 is a set of graphs characterizing skin microbiota. The left panel depicts the relative abundance of the Staphylococcus genus in healthy skin, in skin with atopic dermatitis without lesions (non-lesions), and in skin with atopic dermatitis with lesions. In the lower graphs in the left panel, the relative abundances of two Staphylococcal species, i.e., S. aureus (left graph) and S. epidermidis (right graph) are presented. It is apparent that Staphylococci, including S. aureus and S. epidermidis, are more abundant in skin with atopic dermatitis, with greatest abundance in skin with atopic dermatitis with lesions. In the right panel are graphs showing data establishing the greater relative abundance of four bacterial genera in healthy skin compared to skin with atopic dermatitis (with or without lesions). The four graphs show the relative abundances of the following four genera in healthy skin, skin with atopic dermatitis without lesions, and skin with atopic dermatitis with lesions: upper left graph—Streptococcus; upper right graph—Propionibacterium; lower left graph—Micrococcus; and lower right graph—Corynebacterium. Streptococcus and one additional bacterial genus (Kocuria) showed significant differences between healthy skin and skin with atopic dermatitis without lesions when analyzing the unrarefied pipeline;

FIG. 3 shows shifting in the diversity of skin microbiota from samples taken from 8 locations on the human body of subjects with healthy skin, skin with atopic dermatitis with lesions, and skin with atopic dermatitis without lesions. Samples were subjected to DNA isolation and next generation sequence analysis in conjunction with qPCR to determine the bacterial composition and relative abundances of the skin microbiotas in the 8 locations identified in the figure for each of the three subject types. Results were analyzed and the alpha diversity measure of the Shannon Index showed that the alpha diversity measure was highest for samples taken from healthy skin, an intermediate alpha diversity measure was found for skin with atopic dermatitis without lesions, and the lowest alpha diversity measure was obtained from skin with atopic dermatitis with lesions. The data show that diversity of the skin microbiota decreases as skin develops atopic dermatitis and the diversity continues to decrease as the atopic dermatitis develops lesions.

FIG. 4 is a set of histograms shoring treatment effects on skin microbiota. Only qualified subjects with atopic dermatitis received treatment products during the treatment phase. These qualified subjects were each divided into three treatment groups. Group 1 received a daily 0.5% zinc pyrithione body wash (BCP2ZPT, Group 2 received a vehicle body wash (the vehicle was the same composition as given to Group 1 without the zinc pyrithione, BCP2), and Group 3 received a B7U bar soap. The composition and relative abundances of bacterial genera contributing to the skin microbiota at the onset of the study and after 4 weeks of daily washings are shown for all conditions. “Healthy” is healthy skin; “AD.L” is atopic dermatitis with lesions at onset of the study, “AD.NL” is atopic dermatitis without lesions at the onset of the study, “AD.L4W” is atopic dermatitis with lesions after four weeks of washings, and “AD.NL.4W” is atopic dermatitis without lesions after four weeks of washings. The data shows that zinc pyrithione selectively reduces Staphylococcus abundance in both lesion and non-lesion areas of atopic dermatitis after 4 weeks of washings;

FIG. 5 shows microbiota shift with treatment of atopic dermatitis lesions. The effect of zinc pyrithione body wash on skin microbiota of subjects with atopic dermatitis with lesions was assessed using a standard leg wash assay. Subjects completed a 4-week period of daily leg washes with zinc pyrithione (BCP2ZPT), a B7U bar soap, or a vehicle control (BCP2). Samples were collected at baseline (study onset) and after 4 weeks of leg washings. Results showed that zinc pyrithione selectively reduced the abundance of Staphylococcus on atopic dermatitis lesions, while Propionibacterium, Corynebacterium, and Streptococcus increased in abundance at lesion sites following four weeks of leg washings. Kocuria also increased in the lesion area. The relative increases or decreases were statistically significant at p=0.03, FDR=0.09;

FIG. 6 shows the microbiota shift with treatment of atopic dermatitis without lesions. A study mirroring the study described in the brief description of FIG. 5 was conducted with subjects having atopic dermatitis without lesions substituted for subjects with atopic dermatitis with lesions. The relative abundance of Staphylococcus was measured using 16S rDNA sequencing and qPCR quantification. Subjects performed the standard leg wash using B7U bar soap, vehicle only (BCP2), or zinc pyrithione in BCP2 vehicle (BCP2ZPT). Samples were taken at the onset of the study to establish a baseline and after four weeks of leg washings. Two additional bacterial genera, Streptococcus and Kocuria, were also analyzed and found to respond to zinc pyrithione in BCP2 vehicle with a statistically significant increase in relative abundance using the unrarefied pipeline. The results showed that zinc pyrithione produced a selective reduction in the abundance of Staphylococcus on skin with atopic dermatitis without lesions. Results were statistically significant (p=0.027; PDR>0.05); and

FIG. 7 shows skin microbiota diversity following treatment on subjects with atopic dermatitis with lesions. Subjects with atopic dermatitis with lesions were analyzed for skin microbiota diversity before and after treatment. The subjects were divided into three experimental groups, with one group washing daily for four weeks with BCP2 vehicle body wash (standard leg wash assay), a second group washing daily with B7U bar soap and a third group washing daily with zinc pyrithione in BCP2 vehicle. The composition and relative abundances of bacterial genera in the skin microbiota were measured at the onset of the study to obtain a baseline and after four weeks of daily leg washings. The results showed that zinc pyrithione in BCP2 body wash selectively restored the diversity of the skin microbiota to the diversity level seen in healthy controls.

### DETAILED DESCRIPTION

The disclosure provides materials and methods benefiting man and other animals in increasing the diversity of an amenable skin microbiota, thereby improving the quality and appearance of healthy skin and skin exhibiting atopic dermatitis (with or without lesions), skin dysbiosis or acne, i.e., amenable skin conditions. Healthy skin is non-diseased skin, and it is also skin that does not appear to be dry and/or flaky, such as would be found in an individual with dandruff or with clinically dry skin. Methods of the disclosure are focused on methods of treating skin and not on methods of treating hair (e.g., methods of delivering a bioactive to a hair follicle or infundibulum), or dandruff, and do not involve applications to clinically dry skin. Further, to maximize the availability and, hence, activity of the bioactive components of compositions according to the disclosure, i.e., a zinc compound, a biocompatible surfactant and a lipid, the compositions and methods of use thereof involve the use of free bioactive compounds, i.e., compounds not bound in polymeric form. Preferably, the compositions are also not caged in particulate forms. In general terms, the materials and methods of the disclosure improve the quality and appearance of healthy skin and treat an amenable skin condition of a subject with atopic dermatitis (with or without lesions), skin dysbiosis and/or acne.

A detailed description of the various aspects of the disclosure will be more readily appreciated with an understanding of the following defined terms.

A “zinc-containing compound” or a “zinc compound” is any biologically compatible form of zinc resulting in the uptake of biologically active zinc such as simple zinc salts, e.g., zinc oxide and zinc compounds including zinc monoglycerolate and zinc ionophores, such as zinc pyrithione. The various forms of zinc have been found to have beneficial effects on human skin beyond an anti-microbial action. In particular, zinc compositions have been found to improve the quality of healthy skin, e.g., appearance. Accordingly, the zinc according to the disclosure is not in the form of a dried zinc-polymer aggregate. In other words, the zinc is not sequestered in particles comprising other compounds such as polymers designed to enhance the anti-microbial properties of certain forms of zinc, such as zinc pyrithione.

A “biocompatible surfactant” of the disclosure is any surfactant known in the art to be useful in cleansing skin, including anionic, nonionic, zwitterionic or amphoteric surfactants, provided that the surfactant is also biocompatible, such as mild surfactants like nonionic surfactants but also including sulfate-containing surfactants known to be biocompatible. In its role of cleansing skin, a biocompatible surfactant is defined as a bioactive compound, and not as an excipient. Consistent with this definition, a biocompatible surfactant refers to any mild surfactant or sulfate-containing surfactant that, in the amounts used in the compositions according to the disclosure, do not harm or otherwise have a deleterious effect on the skin. Biocompatible surfactants include anionic surfactants, including sulfate-containing surfactants, nonionic surfactants, amphoteric surfactants, zwitterionic surfactants, and mixtures thereof. Exemplary surfactants include sodium laureth(n) sulfate (SLEnS) surfactants.

The “lipid” according to the disclosure may be any lipid or lipid form known in the art. The lipid may be any of a variety of generally hydrophobic chemical compounds, but a lipid according to the disclosure is not a sphingolipid derivative or a wax. Exemplary lipids include petrolatum, glyceryl monooleate, glycerin, ceramide, cholesterol, a fatty acid, a triglyceride, a phospholipid, or any combination thereof.

“Amenable skin condition” means skin having atopic dermatitis without lesions, skin having atopic dermatitis with lesions, skin exhibiting skin dysbiosis, skin exhibiting acne, or healthy skin. An amenable skin condition is expressly defined to exclude skin dryness or skin characterized as exhibiting, or prone to, dandruff, or to skin infundibula, such as hair follicles.

“Biomarker” refers to any biological molecules (genes, proteins, lipids, metabolites) that can, singularly or collectively, reflect the current or predict future state of a biological system. Thus, as used herein, various biomarkers can be indicators of a quality of skin in terms of skin hydration, among several other properties. Non-limiting examples of biomarkers include inflammatory cytokines, natural moisturizing factors, one or more of keratins 1, 10 and 11, lipids and total protein. The response of skin to treatment with compositions, including personal care compositions for example, can be assessed by measuring one or more biomarkers.

“Multiphase” refers to compositions comprising at least two phases which can be chemically distinct (e.g., a cleansing phase and a benefit phase). Such phases can be in direct physical contact with one another. A personal care composition can be a multiphase personal care composition where phases of the personal care composition can be blended or mixed to a significant degree, but still be physically distinct. In these situations, the physical distinctiveness is undetectable to the naked eye. The personal care composition can be a multiphase personal care composition where the phases are in physical contact and are visually distinct. Visually distinct phases can take many forms, for example, they can appear as striped, marbled, the like.

“Natural moisturizing factor (NMF)” refers to a collection of water-soluble compounds that can be found in the stratum corneum. These compounds comprise about 20-30% of the dry weight of the corneocyte. NMF components absorb water from the atmosphere and combine the water with NMF water content, allowing the outermost layers of the stratum corneum to stay hydrated despite being exposed to external elements. Such NMFs include amino acids, lactic acid, urea, and pyrrolidone carboxylic acid (PCA).

“Package” refers to any suitable container for a personal care composition including but not limited to a bottle, tube, jar, non-aerosol pump, and combinations thereof.

“Personal care composition” refers to compositions intended for topical application to the skin. Personal care compositions can be rinse-off formulations, in which the product can be applied topically to the skin or hair and then subsequently rinsed within seconds to minutes from the skin with water. The product could also be wiped off using a substrate. In either case, it is believed at least a portion of the product is left behind (i.e., deposited) on the skin. The personal care compositions can be extrudable or dispensable from a package. The personal care compositions can be in the form of, for example, a liquid, semi-liquid cream, lotion, gel, or a combination thereof. Examples of personal care compositions can include but are not limited to body wash, moisturizing body wash, shower gels, skin cleansers, cleansing milks, in shower body moisturizer, and cleansing compositions used in conjunction with a disposable cleansing cloth.

“Shannon Index” is given the meaning it has acquired in the art of a measure of biodiversity. The Index is calculated using the formula Shannon Index

$$( H ) = - {\sum }_{i = 1}^{s} {p}_{i} ln {p}_{i} ,$$

wherein p=proportion (n/N) of all individuals (N) in a sample that are a given species (n) and s is the number of species.

“STnS” refers to sodium trideceth(n) sulfate, wherein n can define the average number of moles of ethoxylate per molecule.

“Structured” refers to having a rheology that can confer stability on the personal care composition. A degree of structure can be determined by characteristics determined by one or more of the following methods: Young's Modulus Method, Yield Stress Method, or Zero Shear Viscosity Method or by an Ultracentrifugation Method, described in U.S. Pat. No. 8,158,566, granted on Apr. 17, 2012. A cleansing phase can be considered to be structured if the cleansing phase has one or more following characteristics: (a) Zero Shear Viscosity of at least 100 Pascal-seconds (Pa-s), at least about 200 Pa-s, at least about 500 Pa-s, at least about 1,000 Pa-s, at least about 1,500 Pa-s, or at least about 2,000 Pa-s; (b) A Structured Domain Volume Ratio as measured by the Ultracentrifugation Method, of greater than about 40%, at least about 45%, at least about 50%, at least about 55%, at least about 60%, at least about 65%, at least about 70%, at least about 75%, at least about 80%, at least about 85%, or at least about 90%; or (c) A Young's Modulus of greater than about 2 Pascals (Pa), greater than about 10 Pa, greater than about 20 Pa, greater than about 30 Pa, greater than about 40 Pa, greater than about 50 Pa, greater than about 75 Pa, or greater than about 100 Pa.

“Lather” refers to an aerated foam which results from providing energy to aqueous surfactant mixtures, particularly dilute mixtures.

The phrase “substantially free of” as used herein, unless otherwise specified means that the composition or method comprises less than about 5%, less than about 3%, less than about 1%, or even less than about 0.1% of the stated ingredient. The term “free of” as used herein means that the composition or method comprises 0% of the stated ingredient that is the ingredient has not been added to the personal care composition. However, these ingredients may incidentally form as a byproduct or a reaction product of the other components of the personal care composition.

Disclosed herein are anti-microbial compounds, in combination with a biocompatible surfactant and a lipid in single-phase or multi-phase rinse-off applications which when applied to amenable skin can provide the benefit of improving the appearance of healthy skin or improving a condition such as atopic dermatitis, skin dysbiosis or acne.

The compositions according to the disclosure include an anti-microbial compound, a biocompatible surfactant, and a lipid. These components of the compositions of the disclosure are described in greater detail below.

### Anti-Microbial Compounds

Rinse-off multi-phase cleansing compositions can include an anti-microbial compound. The antimicrobial compounds may include, for example, a zinc-containing compound; carvacrol; helional; menadione; symclairol; alizarin; bardic 2250; chlorhexidine-digluconate; chlorohexenol; 4-isopropyl-3-methylphenol; octopirox, 2-methoxy-1,4-naphthoquinone; 5-hydroxy-1,4-naphthoquinone; 1,2-dodecanediol; phloretin; quercetin hydrate; propyl gallate; methyl 3,4,5-trihydroxybenzoate; octyl gallate; lauryl gallate; ellagic acid tree bark; cinnamic aldehyde; geraniol; thymol; hinokitiol; chrysin; or a combination thereof. The anti-microbial compounds may be included at a level of 0.01 to about 5%, by weight of the cleansing composition.

### a) Zinc-Containing Compounds

A method of enhancing skin appearance can comprise applying a zinc-containing compound such as a zinc ionophore (e.g., a zinc pyrithione) to the skin of an individual. Similarly, a method of treating an amenable skin condition, such as atopic dermatitis (with or without lesions), skin dysbiosis, or acne, can comprise applying a zinc-containing compound to the skin of an individual. Examples of such zinc-containing compounds include, for example, zinc salts. Examples of zinc salts useful herein include the following: zinc aluminate, zinc carbonate, zinc oxide, zinc phosphates, zinc selenide, zinc sulfide, zinc silicates, zinc silicofluoride, zinc borate, zinc hydroxide, zinc hydroxy sulfate, and combinations thereof.

The zinc-containing compound can comprise a zinc salt of 1-hydroxy-2-pyridinethione (known as “zinc pyrithione” or “ZPT”), for example, a mercaptopyridine-Noxide zinc salt. The ZPT can be made by reacting 1-hydroxy2-pyridinethione (i.e., pyrithione acid) or a soluble salt thereof with a zinc salt (e.g., zinc sulfate) to form a zinc pyrithione precipitate, as illustrated in U.S. Pat. No. 2,809,971, and the zinc pyrithione can be formed or processed into platelet ZPT using, for example, sonic energy as illustrated in U.S. Pat. No. 6,682,724.

Zinc pyrithione can take the form of particulates, platelets, or a combination thereof. For example, where the zinc pyrithione is introduced as a particulate, such particulates may have an average particle size from about 0.1 pm to about 20 pm; such particulates may also have an average particle size from about 0.2 pm to about 10 pm.

Other non-limiting zinc-containing compounds include zinc-containing layered materials (“ZLM's”). Examples of zinc-containing layered materials useful herein include zinc-containing layered structures with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLM's) may have zinc incorporated in the layers and/or be components of the gallery ions. Many ZLM's occur naturally as minerals. Common examples include hydrozincite (zinc carbonate hydroxide), basic zinc carbonate, aurichalcite (zinc copper carbonate hydroxide), rosasite (copper zinc carbonate hydroxide) and many related minerals that contain zinc. Natural ZLM's can also occur wherein anionic layer species such as clay-type minerals (e.g., phyllosilicates) contain ion-exchanged zinc gallery ions. All of these natural compounds can also be obtained synthetically or formed in situ in a composition or during a production process. Another common class of ZLM's that are often, but not always, synthetic, is layered double hydroxides, which are generally represented by the formula [M²⁺₁₋ₓM³⁺ₓ(OH)₂]^{x+}A^{m-}_{x/m+}nH₂O and some or all of the divalent ions (M²⁺) would be represented as zinc ions (Crepaldi, E L, Pava, P C, Tronto, J, Valim, J B J. Colloid Interfac. Sci. 2002, 248, 429-42).

Yet another class of ZLMs can be prepared and is called hydroxy double salts (Morioka, H., Tagaya, H., Karasu, M, Kadokawa, J, Chiba, K Inorg. Chem. 1999, 38, 4211-6). Hydroxy double salts can be represented by the general formula [M²⁺₁₋ₓM²⁺₁₊ₓ(OH)_{3(1-y)}]⁺Aⁿ⁻_{(1=3y)/n}nH₂O where the two metal ion may be different; if they are the same and represented by zinc, the formula simplifies to [Zn₁₊ₓ(OH)₂]^{2x+}2x A⁻ nH₂O. This latter formula represents (where x=0.4) and contains common materials such as zinc hydroxychloride and zinc hydroxynitrate. These are related to hydrozincite wherein a divalent anion replaces the monovalent anion. These compounds can also be formed in situ in a composition or in or during a production process. These classes of ZLMs represent relatively common examples of the general category and are not intended to be limiting as to the broader scope of compounds that fit this definition.

Commercially available sources of basic zinc carbonate include Zinc Carbonate Basic (Cater Chemicals: Bensenville, Ill., USA), Zinc Carbonate (Shepherd Chemicals: Norwood, Ohio, USA), Zinc Carbonate (CPS Union Corp.: New York, N.Y., USA), Zinc Carbonate (Elementis Pigments: Durham, UK), and Zinc Carbonate AC (Bruggemann Chemical: Newtown Square, Pa., USA).

Basic zinc carbonate, which also may be referred to commercially as “Zinc Carbonate” or “Zinc Carbonate Basic” or “Zinc Hydroxy Carbonate”, is a synthetic version consisting of materials similar to naturally occurring hydrozincite. The idealized stoichiometry is represented by Zn₅(OH)₆(CO₃)₂ but the actual stoichiometric ratios can vary slightly and other impurities may be incorporated in the crystal lattice.

Suitable examples of pyrithione compounds include zinc pyrithione, sodium pyrithione, pyrithione acid, dipyrithione, chitosan pyrithione, magnesium disulfide pyrithione, and combinations thereof. Pyrithione materials may also include other pyridinethione salts formed from heavy metals such as zinc, tin, cadmium, magnesium, aluminum, and zirconium.

To improve skin appearance and/or improve skin conditions amenable to treatment with the disclosed composition, a zinc-containing compound is applied to, and rinsed from, the skin of an individual at least once per day for several days. Skin treated with a zinc-containing compound of the disclosure can show improvements in, for example, the appearance of the skin. For example, a zinc-containing compound can be applied at least once per day for about 14 days or more; or at least once per day for about 21 days or more.

The zinc-containing compound can be applied directly to the skin or provided as part of a rinse-off personal care composition, which is further described herein. To achieve the enhanced appearance of the skin or improve skin comprising atopic dermatitis (with or without lesions), skin dysbiosis or acne, from about 0.1 μg/cm² to about 5.0 μg/cm²; from about 0.2 μg/cm² to about 5.0 μg/cm²; from about 0.5 μg/cm² to about 5.0 μg/cm²; from about 1.0 μg/cm² to about 3.0 μg/cm²; of a zinc-containing compound is deposited on the skin. Determination of the amount of zinc-containing compound deposited on the skin can be accomplished using techniques known in the art, such as the Cup Scrub method.

Improvements in skin appearance can be measured using known techniques, including, for example, a Corneometer. For example, typical Corneometer units range from about 15-20, wherein the higher the value the higher the level of skin moisturization; and the lower the value, the lower the level of moisturization. Methods for using a Corneometer are described below. Once a zinc-containing compound (e.g., zinc pyrithione) is applied to an amenable skin surface of an individual, a measurement can be taken at predetermined time intervals to evaluate the effectiveness of the zinc-containing compound for improving the appearance of the skin, or for treating a skin with atopic dermatitis (with or without lesions), skin dysbiosis or acne.

For example, measurements taken 3 hours, 24 hours, or 48 hours after the zinc-containing compound has been applied to the skin demonstrate that zinc-containing compounds deposited on the skin can provide vast improvements to amenable skin conditions or to skin appearance. In fact, a Corneometer shows that about 3 hours after the 21^{st} application of the zinc-containing compound to the skin there is readily detectable improvement in skin appearance and/or an amenable skin condition (e.g., at least 0.3 Corneometer units).

Improvements in an amenable skin condition (atopic dermatitis (with or without lesions), skin dysbiosis, acne, or the appearance of healthy skin) can be measured and/or monitored through the use of biomarkers. In particular, natural moisturizing factors (NMFs) constitute an exemplary biomarker that can be detected through methods described herein. One suitable method of obtaining biological samples for measurement of skin NMFs is the application of tape to an epithelium. Any type of tape, including any type of medical tape, is suitable for use in obtaining biological samples of epithelia. This technique is well known in the art and is relatively simple to implement. The technique involves application of tape to the epithelial tissue, typically skin, followed by removal of the tape therefrom. The biomarker analytes obtained from the epithelial tissue and present on the tape can then be removed from the tape in any fashion that preserves the biomarker analytes for suitable detection and measurement assays. Suitable biomarkers and testing procedures for NMFs are described in U.S. patent application Ser. No. 13/007,630.

While improvements in skin condition can be measured using a Corneometer or biomarkers are exemplary approaches to measuring and/or monitoring an amenable skin condition such as atopic dermatitis, skin dysbiosis, acne, or the appearance of healthy skin, but other suitable measuring and monitoring methods are available that focus on other properties of amenable skin conditions. For example, change in an amenable skin condition can be measured and/or monitored by assessing the visual dryness of skin, trans-epidermal water loss (TEWL), total protein in the stratum corneum layer of skin, involucrin level in skin, HSA level in skin, lipid level in skin, relative differentiation of Keratins 1, 10 and 11 in skin, inflammatory cytokine (e.g., IL-1α, IL-1rα, IL-8) level in skin, and histamine level in skin.

### Biocompatible Surfactants

A method of enhancing skin appearance also comprises at least one biocompatible surfactant. The biocompatible surfactant can be anionic, non-ionic, amphoteric, zwitterionic, or a combination thereof. One example of a biocompatible surfactant is sodium laureth(n) sulfate, hereinafter SLEnS, wherein n defines the average moles of ethoxylation. In this context, the n variable can range from about 1 to about 3. Another example of a biocompatible surfactant is the anionic surfactant of sodium trideceth(n) sulfate, where n is from 0.5 to 2.7. Additional exemplary surfactants for use in the compositions of the disclosure include cocamidopropyl betaine, sodium lauroamphoacetate, sodium trideceth sulfate, and sodium cocoyl isethionate. The rinse-off personal care composition can include at least one of an amphoteric surfactant and a zwitterionic surfactant. Suitable amphoteric or zwitterionic surfactants include those described in U.S. Pat. Nos. 5,104,646 and 5,106,609.

Amphoteric surfactants can include those that can be broadly described as derivatives of aliphatic secondary and tertiary amines in which an aliphatic radical can be straight or branched chain and wherein an aliphatic substituent can contain from about 8 to about 18 carbon atoms such that one carbon atom can contain an anionic water solubilizing group, e.g., carboxy, sulfonate, sulfate, phosphate, or phosphonate. Examples of compounds falling within this definition can be sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to U.S. Pat. No. 2,658,072, N-higher alkyl aspartic acids such as those produced according to U.S. Pat. No. 2,438,091, and products described in U.S. Pat. No. 2,528,378. Other examples of amphoteric surfactants can include sodium lauroamphoacetate, sodium cocoamphoacetate, disodium lauroamphoacetate disodium cocodiamphoacetate, and mixtures thereof. Amphoacetates and diamphoacetates can also be used.

Zwitterionic surfactants suitable for use can include those that are broadly described as derivatives of aliphatic quaternary ammonium, phosphonium, and sulfonium compounds, in which aliphatic radicals can be straight or branched chains, and wherein an aliphatic substituent can contain from about 8 to about 18 carbon atoms such that one carbon atom can contain an anionic group, e.g., carboxy, sulfonate, sulfate, phosphate, or phosphonate. Other zwitterionic surfactants can include betaines, including cocoamidopropyl betaine.

### Lipids

The compositions of the disclosure also comprise a lipid. Lipids according to the disclosure include any of a variety of natural or synthetic oils, fats or other generally hydrophobic compounds recognized in the art as lipids. Exemplary lipids include glycerides suitable for use as hydrophobic skin benefit agents, including castor oil, safflower oil, corn oil, walnut oil, peanut oil, olive oil, cod liver oil, almond oil, avocado oil, palm oil, sesame oil, soybean oil, vegetable oils, sunflower seed oil, vegetable oil derivatives, coconut oil and derivatized coconut oil, cottonseed oil, derivatized cottonseed oil, jojoba oil, cocoa butter, petrolatum, mineral oil, and combinations thereof.

Non-limiting examples of alkyl esters suitable for use as lipid skin benefit agents herein include isopropyl esters of fatty acids and long chain esters of long chain (i.e., C10-C24) fatty acids, e.g., cetyl ricinoleate, non-limiting examples of which include isopropyl palmitate, isopropyl myristate, cetyl ricinoleate, and stearyl ricinoleate. Other examples include hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use as hydrophobic skin benefit agents herein include oleyl myristate, oleyl stearate, oleyl oleate, and combinations thereof.

Non-limiting examples of polyglycerin fatty acid esters suitable for use as lipid skin benefit agents include decaglyceryl distearate, decaglyceryl diisostearate, decaglyceryl monomyristate, decaglyceryl monolaurate, hexaglyceryl monooleate, and combinations thereof.

Non-limiting examples of lanolin and lanolin derivatives suitable for use as lipid skin benefit agents include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, and combinations thereof.

Non-limiting examples of silicone oils suitable for use as lipid skin benefit agents include dimethicone copolyol, dimethylpolysiloxane, diethylpolysiloxane, mixed Cl-C30 alkyl polysiloxanes, phenyl dimethicone, dimethiconol, and combinations thereof. Non-limiting examples of silicone oils useful herein are described in U.S. Pat. No. 5,011,681.

Still other suitable hydrophobic skin benefit agents include milk triglycerides (e.g., hydroxylated milk glyceride) and polyol fatty acid olyesters.

### Rinse-Off Personal Care Compositions

As discussed above, anti-microbial compounds, like zinc-containing compounds (e.g., zinc pyrithione), can be applied to the skin through a rinse-off personal care composition. Suitable zinc-containing compounds are disclosed above. A rinse-off personal care composition can be a single-phase composition or a multi-phase composition. The rinse-off personal care composition can also involve a single-phase application or a multi-phase application that includes a cleansing phase and a benefit phase. The cleansing phase and/or benefit phase can include the anti-microbial compound, like a zinc-containing compound (e.g., zinc pyrithione). The cleansing phase can also include, for example, various biocompatible surfactants as described herein. The benefit phase can comprise an effective amount of a lipid. The cleansing phase and the benefit phase can be blended, and/or patterned. The rinse-off personal care composition is in a form that is not an emulsion.

The rinse-off personal care composition can comprise at least about 0.1%, by weight of the rinse-off personal care composition, of a zinc-containing compound (e.g., zinc pyrithione). The rinse-off personal care composition can also comprise from about 0.2% to about 1.0%, by weight of the rinse-off personal care composition, of a zinc-containing compound (e.g., zinc pyrithione). The rinse-off personal care composition can also comprise about 0.5%, by weight of the rinse-off personal care composition, of a zinc-containing compound (e.g., zinc pyrithione).

A. Cleansing Phase

A cleansing phase of a multi-phase composition can comprise a zinc-containing compound (e.g., a zinc ionophore such as zinc pyrithione), as described herein. Further, the cleansing phase includes at least one biocompatible surfactant. For example, the cleansing phase can include an aqueous structured surfactant that is biocompatible. The concentration of the structured surfactant in the personal care composition can range from about 1% to about 20%, by weight; from about 2% to about 15%, by weight; and from about 5% to about 10%, by weight of the personal care composition.

Such a structured surfactant can include sodium trideceth(n) sulfate, hereinafter STnS, wherein n defines the average moles of ethoxylation. The n variable can range from about 0 to about 3. The n variable can also range from about 0.5 to about 2.7, from about 1.1 to about 2.5, from about 1.8 to about 2.2, or n can be about 2. When n can be less than 3, STnS can provide improved stability, improved compatibility of benefit agents within the rinse-off personal care compositions, and increased mildness of the rinse-off personal care compositions. These benefits of STnS are disclosed in U.S. patent application Ser. No. 13/157,665.

Further, the cleansing phase can comprise a structuring system, wherein the structuring system can comprise, optionally, a non-ionic emulsifier and an electrolyte. Suitable emulsifiers and electrolytes are described in U.S. patent application Ser. No. 13/157,665. The rinse-off personal care composition can be optionally free of sodium lauryl sulfate (SLS), and can comprise at least a 70% lamellar structure.

Suitable surfactants or co-surfactants that can generally be used in a cleansing phase for a rinse-off personal care composition are described above and/or in McCutcheon's: Detergents and Emulsifiers North American Edition (Allured Publishing Corporation 1947) (1986), McCutcheon's, Functional Materials North American Edition (Allured Publishing Corporation 1973) (1992) and U.S. Pat. No. 3,929,678.

The cleansing phase may also include water in a range of about 0% to about 95%, by weight of the composition.

B. Benefit Phase

As noted herein, rinse-off personal care compositions can include a benefit phase. The benefit phase can be hydrophobic and/or anhydrous. The benefit phase can also be substantially free of or free of surfactant.

The benefit phase can also include a benefit agent comprising a lipid. In particular, the benefit phase can comprise from about 0.1% to about 50%, by weight of the rinse-off personal care composition, of the benefit agent. The benefit phase can also include from about 0.5% to about 20%, by weight of the rinse-off personal care composition, of the benefit agent. Examples of the benefit agent can include artificial sweat, castor oil, olive oil, oleic acid, Sefose® 1618S, Sefose® 1618U, petrolatum, glyceryl monooleate, mineral oil, natural oils (e.g., soybean oil), and mixtures thereof. Other suitable benefit agents are described in U.S. patent application Ser. No. 13/157,665.

As noted herein, the benefit phase can include a zinc-containing and/or pyrithione material (e.g., zinc pyrithione), as described above. Examples of such zinc-containing materials can include, for example, zinc salts. Examples of zinc salts useful can include the following: zinc aluminate, zinc carbonate, zinc oxide, zinc phosphates, zinc selenide, zinc sulfide, zinc silicates, zinc silicofluoride, zinc borate, zinc hydroxide, zinc hydroxy sulfate, and combinations thereof. The benefit phase can also include additional ingredients as described below.

The benefit phase can typically comprise one or more benefit agents, as set forth above. The benefit phase can comprise from about 0.1% to about 50%, by weight of the rinse-off personal care composition, of the benefit agent in the form of a lipid.

Additional ingredients can optionally be added to the rinse-off personal care composition for treatment of the skin, or to modify the aesthetics of the rinse-off personal care composition as is the case with perfumes, colorants, dyes or the like. Optional materials useful in products herein can be categorized or described by their cosmetic and/or therapeutic benefit or their postulated mode of action or function. However, it can be understood that actives and other materials useful herein can, in some instances, provide more than one cosmetic and/or therapeutic benefit or function or operate via more than one mode of action. Therefore, classifications herein can be made for convenience and cannot be intended to limit an ingredient to particularly stated application or applications listed. A precise nature of these optional materials, and levels of incorporation thereof, will depend on the physical form of the composition and the nature of the cleansing operation for which it is to be used. Optional materials can usually be formulated at about 6% or less, about 5% or less, about 4% or less, about 3% or less, about 2% or less, about 1% or less, about 0.5% or less, about 0.25% or less, about 0.1% or less, about 0.01% or less, or about 0.005% or less of the rinse-off personal care composition.

To further improve stability under stressful conditions such as high temperature and vibration, densities of separate phases can be adjusted such that they can be substantially equal. To achieve this, low density microspheres can be added to one or more phases of the rinse-off personal care composition. Examples of rinse-off personal care compositions that comprise low density microspheres are described in U.S. Patent Publication No. 2004/0092415A1.

Other non-limiting optional ingredients that can be used in the personal care composition can comprise an optional benefit component that can be selected from the group consisting of thickening agents; preservatives; fragrances; chelators (e.g., such as those described in U.S. Pat. No. 5,487,884 issued to Bisset, et al.); sequestrants; vitamins (e.g., Retinol); vitamin derivatives (e.g., tocophenyl acetate, niacinamide, panthenol); sunscreens; desquamation actives (e.g., such as those described in U.S. Pat. Nos. 5,681,852 and 5,652,228 issued to Bisset); anti-wrinkle/anti-atrophy actives (e.g., N-acetyl derivatives, thiols, hydroxyl acids, phenol); anti-oxidants (e.g., ascorbic acid derivatives, tocophenol) skin soothing agents/skin healing agents (e.g., panthenoic acid derivatives, aloe vera, allantoin); skin lightening agents (e.g., kojic acid, arbutin, ascorbic acid derivatives) skin tanning agents (e.g., dihydroxyacetone); anti-acne medicaments; essential oils; sensates; pigments; colorants; pearlescent agents; interference pigments (e.g., those disclosed in U.S. Pat. Nos. 6,395,691, 6,645,511, 6,759,376, 6,780,826) particles (e.g., talc, kolin, mica, smectite clay, cellulose powder, polysiloxane, silicas, carbonates, titanium dioxide, polyethylene beads) hydrophobically modified non-platelet particles (e.g., hydrophobically modified titanium dioxide and other materials described in U.S. Pat. Pub. No. 2006/0182699A) and mixtures thereof. The multiphase personal care composition can comprise from about 0.1% to about 4%, by weight of the rinse-off personal care composition of hydrophobically modified titanium dioxide. Other such suitable examples of such skin actives are described in U.S. patent application Ser. No. 13/157,665.

Other optional ingredients can be most typically those materials approved for use in cosmetics and that are described in the CTFA Cosmetic Ingredient Handbook, Second Edition, The Cosmetic, Toiletries, and Fragrance Association, Inc. 1988, 1992.

### Exemplary Procedures

A. Cup Scrub Procedure

As noted herein, the Cup Scrub Procedure can be used to assist in determining how much zinc-containing compound (e.g., zinc ionophore such as zinc pyrithione) is deposited onto the skin of an individual. In performing the procedure, test subjects first wet the volar forearm surface under running water (flow=4.5 L/min, temp=35-38° C.) for approximately 15 seconds. Next, test subjects receive a dose of 1 mL of body wash (via disposable syringe) to the volar forearm surface. The subjects proceed to generate lather on the volar forearm by rubbing the applied body wash with their opposite hand for approximately 15 seconds. Following the 15-second lathering process, the lather is allowed to sit undisturbed on the skin for an additional 15 seconds. At the end of the 15-second wait (30 seconds after the start of the lathering process), the subjects rinse the arm for approximately 10 seconds, allowing the running water to contact the proximal volar forearm surface and cascade down (toward the distal surface). Following the rinse, the subjects use a paper towel to pat the surface dry.

The next part of the procedure involves a 2-cm diameter glass cylinder containing a bead of silicone caulking on a skin contact edge that will be pressed firmly against a skin surface to prevent leakage of an extraction fluid. One mL of the extraction solvent can be pipetted into the glass cylinder. To determine how much zinc pyrithione is deposited, for example, the extraction solvent can be 80:20 0.05 M EDTA:Ethanol. While using a transfer pipette or glass rod, an entire area within the glass cylinder can be scrubbed for about 30 seconds using moderate pressure. The solution can be removed and pipetted into a labeled glass sample vial. The Cup Scrub Procedure can be repeated using fresh extraction solution, which will be pooled with the initial extraction in the labeled vial.

After each use, the glass cylinder and rod are cleaned. For example, each cylinder and rod can be immersed in dilute Dawn® solution and scrubbed with a finger or soft bristle brush. The cylinders and rods can then be immersed in isopropyl alcohol (IPA). Finally, cylinders and rods are wiped dry, e.g., with a Kimwipe or other lint free tissue, to remove any visible residue. Scrub solutions can be changed at the end of each day or when any visible layer of residue can be found in the bottom thereof. Further, samples can be stored at 4° C. (±3° C.) until the samples are submitted for HPLC analysis. HPLC analysis is then used to determine the amount of deposition. The free pyrithione in solution is then derivatized with 2-2′-Dithiopyridine, and subsequently analyzed via HPLC utilizing UV detection. The results are reported as μg of zinc pyrithione per mL of solution.

B. Monitoring Biomarkers: Natural Moisturizing Factors (NMFs)

Biomarkers that can be indicative of skin health can be measured to evaluate changes on one or more surfaces of epithelial tissue of a subject exposed to a product according to the disclosure. Thus, biomarkers can allow for a relatively simple, efficient and quick determination of the usefulness of a product for providing one or more benefits to skin, or for monitoring changes in the skin upon or after exposure of the skin to a composition according to the disclosure.

Samples of epithelial tissue can be obtained to collect and analyze biomarker analytes. Non-limiting examples of suitable techniques for obtaining samples include application of tape, rinsing by lavage, biopsy, swabbing, scraping, blotting and combinations thereof. Whichever technique is used to obtain a sample, it should be one where the biomarkers obtained are those present on the surface and/or in the epithelial tissue, and not those in any of the underlying non-epithelial tissue, such as muscle.

A method of obtaining epithelial tissue is by application of tape, such as, but not limited to, any type of medical tape. A technique for applying tape can be as straightforward as applying tape to the skin and then removing it. Biomarker analytes obtained from the skin and present on the tape can be removed from the tape by any technique known in the art that preserves the biomarker analytes for suitable detection and measurement assays. Examples of tapes can include, but are not limited to, D-squame Tape® and SEBUTAPE®, both of which are available from CuDerm Corporation, Dallas, Tex., USA; and Transpore® tape, which is available from the 3M Company, of Minnesota USA.

Biomarker analytes can be present in test and control samples and can be identified using one or more techniques known in the art. Detection techniques such as antibody-based binding methodologies, nucleotide probe-based specific hybridization assays, highly specific chemical tagging using markers, dyes, and other colorimetric and fluorometric probes and assays, as well as enzyme-linked production of detectable labeled compounds can be used to detect and measure biomarker analytes. In some non-limiting examples, biomarker analytes include inflammatory cytokines, natural moisturizing factors (NMFs), keratin 1, keratin 10, keratin 11, lipids and total protein.

Exemplary biomarkers are natural moisturizing factors or NMFs. Examples of NMFs include amino acids, lactic acid, urea, and pyrrolidone carboxylic acid (PCA), and more particularly include Trans-Urocanic Acid, Citrulline, Glycine, Histidine, Ornithine, Proline, 2 Pyrrolidone 5 Acid, and Serine. As set forth above, effectiveness of treatment with a composition of the disclosure can be evidenced by an increase in the amount of NMFs. NMFs can be measured to detect improvement in skin appearance or reduction in the progression of an amenable skin condition in the form of atopic dermatitis (with or without lesions), skin dysbiosis or acne. Such methodologies are further described in U.S. patent application Ser. No. 13/007,630.

To measure NMF values, the following methods can be used. Tape strips (D-Squame) from subjects are placed into polypropylene tubes and mixed, by vortex or sonication, with acidified water to extract relevant amino acid-related NMFs (glycine, histidine, proline, serine, urocanic acid, citrulline ornithine and 2-pyrrolidone5-carboxylic acid). Extracts from the tape strips are spiked with stable-isotope internal standards of each NMF and then analyzed by gradient reversed-phase high-performance liquid chromatography with tandem mass spectrometry using multiple-reaction-monitoring. Combined standards for the NMFs are prepared over the required concentration range, spiked with the stable-isotope internal standards, and analyzed along with the samples. The response ratio of each standard (response of standard/response of internal standard) for each NMF is plotted versus the standard concentration to generate a regression curve for each of the NMFs. The concentration of each NMF in the extracts is then determined by interpolation from the appropriate regression standard curve.

The following examples illustrate exemplary methods of the disclosure. Example 1 provides an analysis of the skin microbiota of a subject with atopic dermatitis. Example 2 discloses a comparison of the skin microbiotas of a subject with atopic dermatitis and a healthy subject. Example 3 shows the changes in diversity of skin microbiotas as healthy skin develops atopic dermatitis. Example 4 reveals the measuring/monitoring of changes in the skin microbiota as an amenable skin condition such as atopic dermatitis is treated with a composition according to the disclosure.

### EXAMPLES

### Example 1

Microbiota Analysis of the Skin of a Subject with Atopic Dermatitis

The microbial load associated with the microbiota of skin having atopic dermatitis is heavier than the microbial load associated with healthy skin. Use of zinc pyrithione in a body wash lowers the total bacterial level.

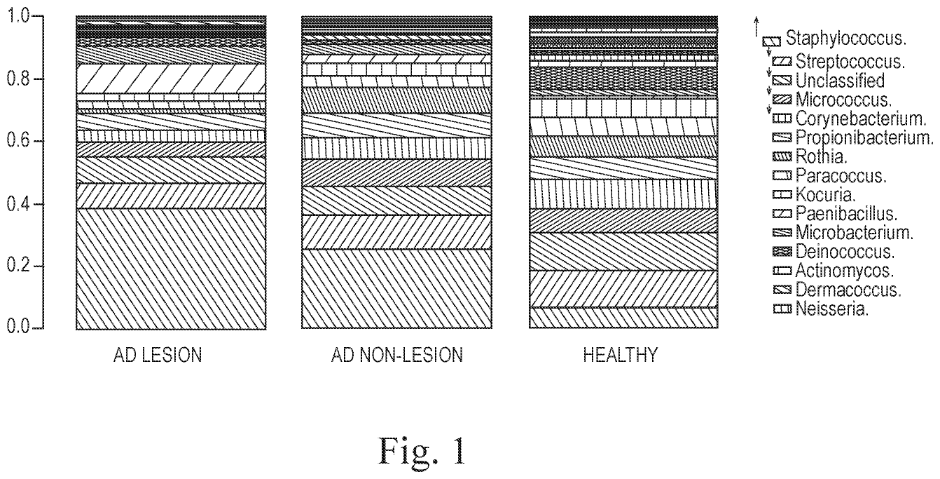

Samples of microbiotas from skin with atopic dermatitis were subjected to sequence analyses to identify the various genera present on the skin and to measure relative abundances. In a first sequencing round, read lengths of 150-800 bp were obtained using the Closed OTU picking method. The Mega-blast search engine was used to interrogate the GG-13-8-91 database. The second sequencing round yielded read lengths of 250-600 bp using the Cosed OTU picking method. The Bowtie2 search engine was used to again interrogate the GG-13-8-91 database. The third sequencing round involved read lengths of 250-600 bp being obtained using the Cosed OTU picking method. The Bowtie2 search engine was used to interrogate the GG-13-8-97 database. Results are shown in FIG. 1.

The data show that Staphylococcus is the predominant bacterial genus present in the skin microbiota of subjects with atopic dermatitis. Further, the data establish that the skin microbiota associated with atopic dermatitis comprises more than fourteen bacterial genera.

### Example 2

### Comparison of Atopic Dermatitis Skin Microbiota to Healthy Skin Microbiota

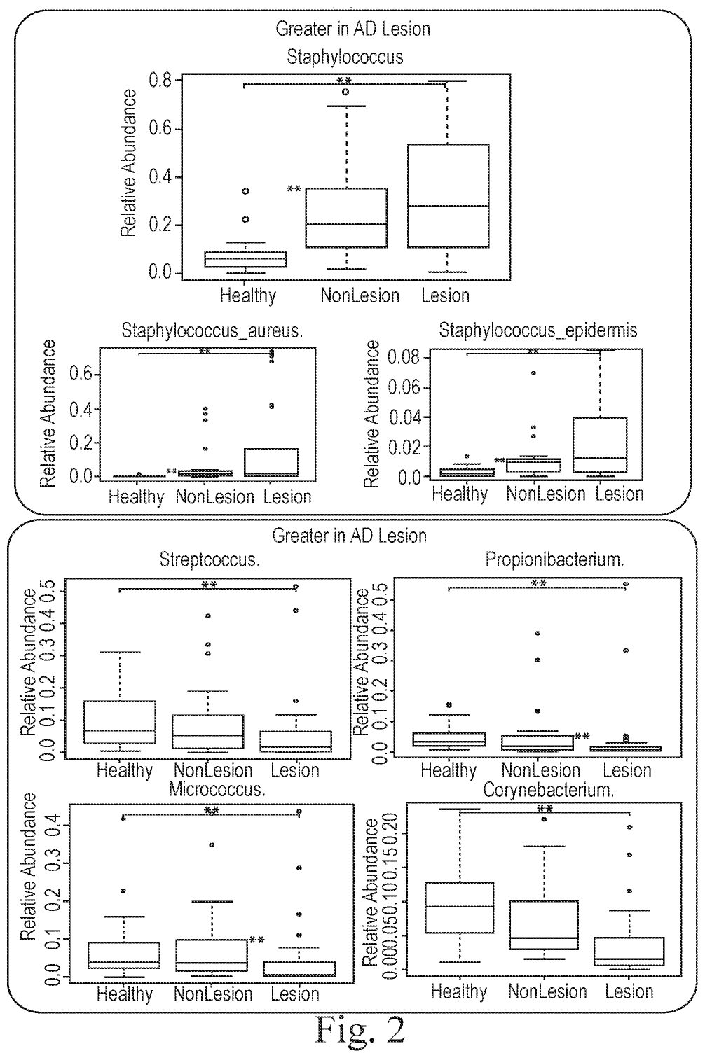

A detailed comparison of the skin microbiotas from healthy subjects and subjects with atopic dermatitis revealed several noteworthy features. As noted in FIG. 2, several Staphylococcal species are present in greater proportion in the skin microbiota of atopic dermatitis subjects compared to the skin microbiota of healthy subjects, and the discrepancy is more pronounced when comparing subjects with atopic dermatitis lesions to healthy subjects than when comparing atopic dermatitis subjects without lesions to healthy subjects. Staphylococcus aureus and Staphylococcus epidermidis are each present in greater relative abundance in atopic dermatitis skin microbiotas than in healthy skin microbiotas, and the relative abundances of each of these Staphylococcal species is greater in skin microbiotas of subjects with atopic dermatitis lesions than atopic dermatitis subjects without lesions.

In contrast, several bacterial genera exhibit greater relative abundance in healthy skin microbiota relative to skin microbiotas of subjects with atopic dermatitis, regardless of whether the atopic dermatitis subjects exhibit lesions or not. Bacterial genera exhibiting this pattern of relative abundance include Streptococcus, Propionibacterium, Micrococcus and Corynebacterium. Mirroring the observations with the Staphylococcal species, each of the Streptococcus, Propionibacterium, Micrococcus and Corynebacterium genera are present in greater relative abundance in atopic dermatitis subjects with lesions than in atopic dermatitis subjects lacking lesions.

### Example 3

### Bacterial Diversity During Atopic Dermatitis Development

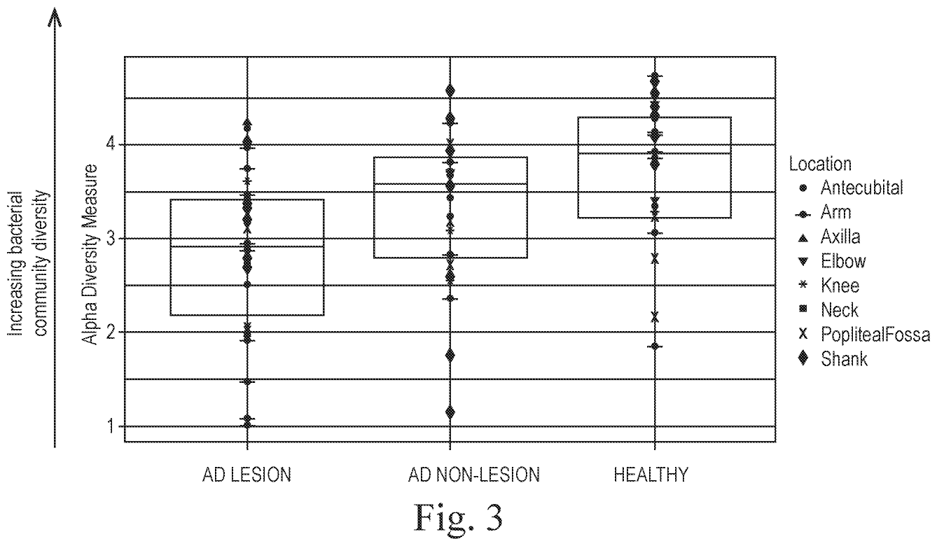

In the course of developing atopic dermatitis, healthy skin changes to reveal the characteristic inflammatory indicators of atopic dermatitis, eventually leading to skin lesions. The data obtained from sequence analyses of skin microbiota samples (see Example 1) taken from the following skin regions of subjects were subjected to statistical analysis: the antecubital region, the arm, the axilla, the elbow, the knee, the neck, the popliteal fossa, and the shank of the leg. The statistical analyses revealed alpha diversity values based on the Shannon Index. As shown in FIG. 3, the alpha diversity measures of the bacterial diversity of atopic dermatitis skin microbiotas showed that diversity was greatest in healthy skin, with reduced diversity in atopic dermatitis skin microbiotas lacking lesions, and the lowest diversity in microbiotas from atopic dermatitis subjects exhibiting lesions. Also apparent is that the pattern of relative diversities remained the same across all skin sampling locations.

### Example 4

### Skin Microbiota-Based Monitoring of Skin Disorder (Atopic Dermatitis) Treatment

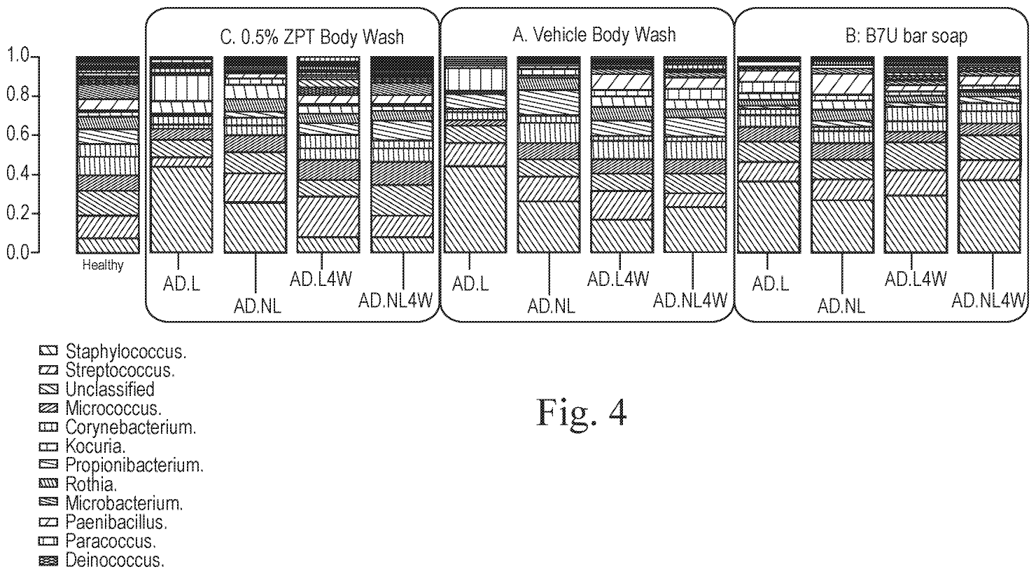

The compositions of the disclosure comprise a zinc ionophore as a significant component, with zinc pyrithione being an exemplary zinc ionophore. A study was conducted of the effect on skin microbiota of single washes or four-week wash periods of either a 0.5% zinc pyrithione body wash, a vehicle body wash, or a B7U bar soap wash. The relative abundances of 11 identified bacterial genera were monitored, along with a category of unclassified microbes. The 11 identified bacterial genera were Staphylococcus, Streptococcus, Micrococcus, Corynebacterium, Kocuria, Propionibacterium, Rothia, Microbacterium, Paenibacillus, Paracoccus, and Deinococcus. Skin samples were obtained from healthy subjects (designated “healthy” in FIG. 4) as wells as subjects with atopic dermatitis lesions receiving a single wash (“AD.L”), subjects with atopic dermatitis but no visible lesions receiving a single wash (“AD.NL”), subjects with atopic dermatitis lesions receiving a four-week wash period (“AD.L4W”), and subjects with atopic dermatitis but no lesions receiving a four-week wash period (“AD.NL4W”). The data shown in FIG. 4 establish that the 0.5% zinc pyrithione body wash led to significant changes in the relative abundances of the 11 identified bacterial genera over the four-week wash period. Notable among the changes in subjects with atopic dermatitis lesions over the four-week wash period were significant decreases in the relative abundances of Staphylococcus, Microbacterium, and Paenibacillus, and significant increases in the relative abundances of Streptococcus, Micrococcus, Corynebacterium, Kocuria, Propionibacterium, Rothia, Paracoccus and Deinococcus. For atopic dermatitis patients not exhibiting lesions, however, the pattern of skin microbiota changes differed. Over the four-week wash period, the relative abundances of Staphylococcus, Streptococcus, Kocuria, Microbacterium, and Paenibacillus, decreased, and the relative abundances of Micrococcus, Corynebacterium, Propionibacterium, Paracoccus, and Deinococcus increased. There was a slight decrease in the relative abundance of Rothia over the four-week wash period for subjects with atopic dermatitis lacking visible lesions.

The changes in skin microbiota diversity, as measured by the relative abundances of the identified bacterial genera, were more dramatic for subjects completing the four-week wash study using the 0.5% zinc pyrithione body wash compared to either the B7U bar soap or to a body wash with vehicle alone. In addition, a comparison of the relative abundances of the bacterial genera to healthy skin controls revealed that subjects with atopic dermatitis altered their skin microbiota diversity in a manner that brought them closer to the microbiota diversity of healthy skin over the four-week wash period, with subjects having atopic dermatitis but no lesions approaching the skin microbiota diversity of healthy skin more closely. The data show that zinc pyrithione is effective at the selective modulation or adjustment of bacterial genera relative abundances and that the zinc ionophore shifts the skin microbiota composition to one that more closely matches healthy skin, a sign that the therapeutic has a beneficial effect in modulating the skin microbiota of subjects with sound, but unhealthy skin, such as subjects with such skin disorders as atopic dermatitis, skin dysbiosis, or acne.

Another study was conducted to specifically assess the effect of a zinc ionophore, i.e., zinc pyrithione, on the skin microbiota diversity of atopic dermatitis lesions using standard body wash protocol well-known in the art. The washes were carried out on a daily basis over a four-week period and 16S rDNA sequencing was used to identify bacterial genera while qPCR amplification was used to quantify the abundance of the genera. Measurements were taken at baseline and after the four-week wash period. Controls involved subjects washing with B7U bar soap or the BCP2 vehicle alone. BCP2 vehicle comprises cocoamidopropyl betaine (2.45%), sodium chloride (4.03%), trideceth-3 (1.31%), guar hydroxypropyltrimonium chloride (0.43%), xanthan gum (0.19%), acrylate/C10-C30 alkyl acrylates crosspolymer (0.03%), sodium benzoate (0.27%), citric acid (50% in DI water to pH=5.7), preservative (0.04%), perfume (0.25%), and water and minors (Q.S.); where appropriate, zinc pyrithione (0.5%), sodium trideceth-2 sulfate (8.21%), petrolatum (14.70%), and glyceryl monooleate (0.30%) can be included. The compositions of the BCP2 and BCP2ZPT compositions are listed below. The compositions can be prepared by first adding water into a main mixing container with continuous mixing. Then add sodium chloride, xanthan gum, guar hydroxypropyltrimonium chloride, sodium trideceth sulfate, cocoamidopropyl betaine. Create a premix with Trideceth-3 and Acrylates/C10-C30 Alkyl acrylates crosspolymer. Add the premix into the main mixing container. Add sodium benzoate and EDTA. Then, adjust pH to 5.7. Add kathon and perfume. Add ZPT in the BCP2-ZPT product.

| | BCP2 | BCP2ZPT |
|---|---|---|
| Cleansing Phase | | |
| Sodium Trideceth (n = 2) | 8.21% | 8.21% |
| Sulfate | | |
| Cocoamidopropyl betaine | 2.45% | 2.45% |
| Sodium chloride | 4.03% | 4.03% |
| Trideceth-3 | 1.31% | 1.31% |
| Guar Hydroxypropyl- | 0.43% | 0.43% |
| trimonium Chloride | | |
| Sodium Benzoate | 0.27% | 0.27% |
| Citric Acid | to pH = | to pH = |
| | 5.7 | 5.7 |
| Xanthan Gum | 0.19% | 0.19% |
| Acrylates/C10-C30 Alkyl | 0.03% | 0.03% |
| Acrylates crosspolymer | | |
| Kathon CG | 0.03% | 0.03% |
| ZPT | — | 0.5% |
| Perfume | 0.2% | 0.2% |
| Others (water and minors) | QS | QS |
| Benefit Phase | | |
| Petrolatum | 14.7% | 14.7% |
| Glyceryl monooleate | 0.3% | 0.3% |

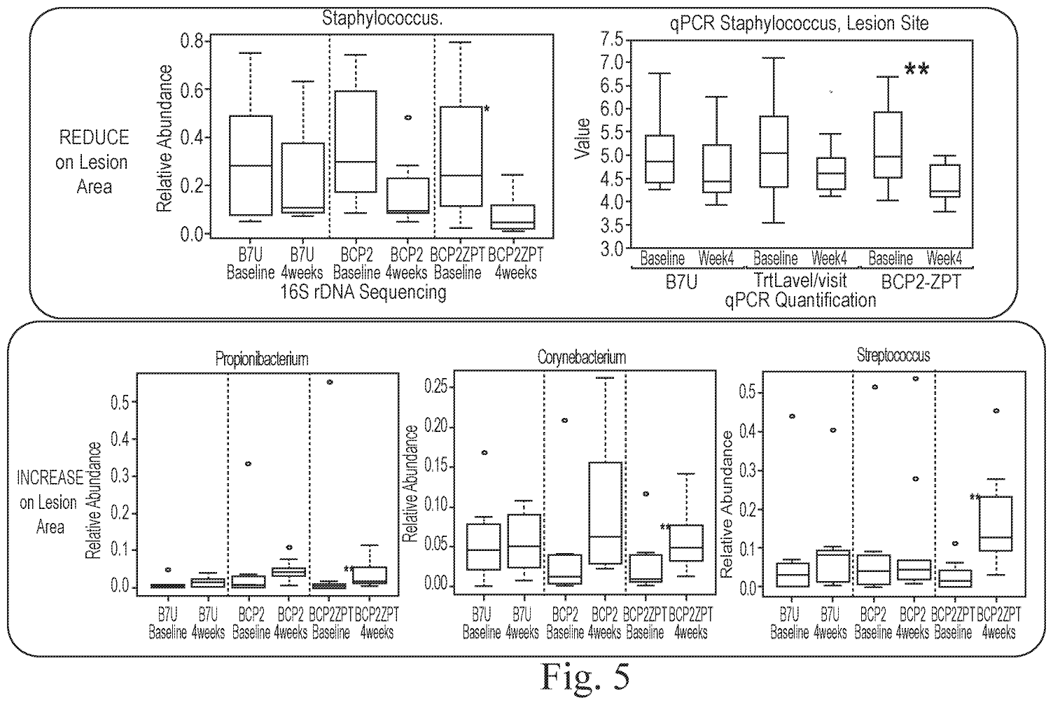

The Results shown in FIG. 5 reveal that the specific treatment with the zinc ionophore in the form of zinc pyrithione over the four-week wash period led to a significant decrease (p=0.03, FDR=0.09) in the relative abundance of Staphylococcus and a significant relative increase in the abundances of Propionibacterium, Corynebacterium and Streptococcus. Additional data established that the relative abundances of Streptococcus and another bacterial genus, i.e., Kocuria, differed significantly between non-lesioned atopic dermatitis skin (sound but unhealthy skin) and healthy skin.

A related experiment assessing the treatment effects of a zinc ionophore on skin microbiota diversity in subjects with atopic dermatitis without lesions was performed to assess the effects of the therapeutic in this context. The experimental design involved daily body washes of subjects with atopic dermatitis lacking visible lesions over a four-week period, with subjects engaging in daily washing with either B7U bar soap, vehicle alone (BCP2 base), or BCP2ZPT (which included 0.5% zinc pyrithione). Skin samples were again analyzed for microbiota composition by 16S rDNA sequencing with qPCR to quantify the abundances of the bacterial genera in the skin microbiotas.

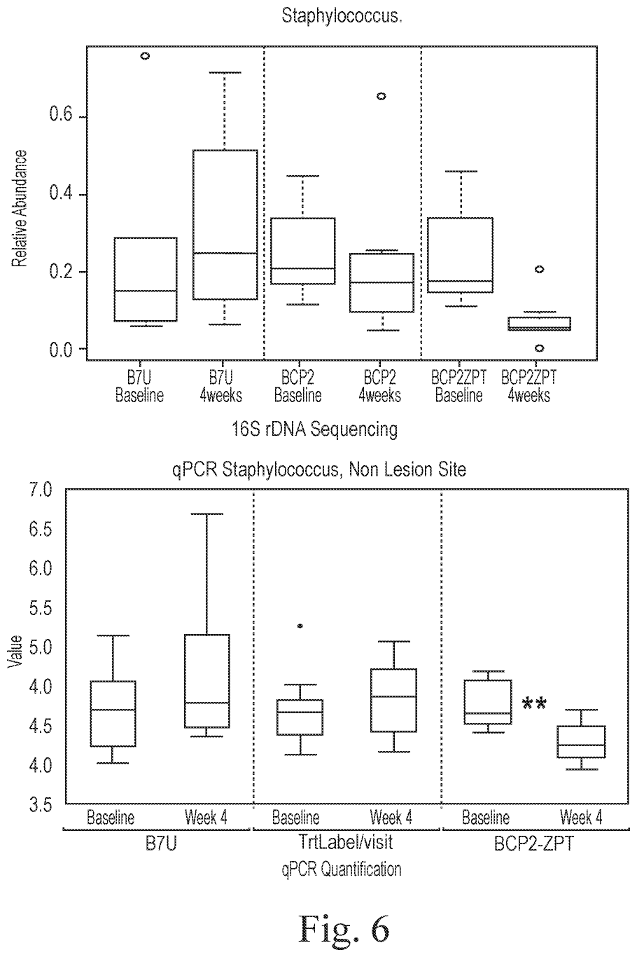

The results are presented in FIG. 6 and show that the zinc ionophore therapeutic in the form of zinc pyrithione significantly reduced the abundance of Staphylococcus (p=0.027, FDR>0.05), whereas four weeks of washing with B7U bar soap or vehicle alone (BPC2) did not significantly reduce the abundance of Staphylococcus on the skin of subjects with atopic dermatitis but no visible lesions. Additional data showed that the four-week washing regimen with the zinc ionophore also led to significant differences in the relative abundances of Streptococcus and Kocuria. It is expected that compositions containing the zinc ionophore, such as compositions containing a zinc ionophore (e.g., zinc pyrithione), a biocompatible surfactant, and a lipid will not only affect the relative abundances of Staphylococcus, Streptococcus and Kocuria, but the additional bacterial genera identified in Example 4 (see FIG. 4).

An experiment was also conducted to assess whether the skin microbiota diversity of lesion areas of subjects with atopic dermatitis could be restored. The experimental design involved a four-week period of daily leg washes with subjects washing with either B7U bar soap, vehicle (BCP2), or BCP2ZPT. Skin containing lesions in the following areas were sampled: antecubital area, arm, elbow, knee, neck, popliteal fossa and the shank of the leg. Results compared the skin microbiota diversity measures obtained at week 0 and week 4. The diversity measures were the alpha values based on the Shannon Index.

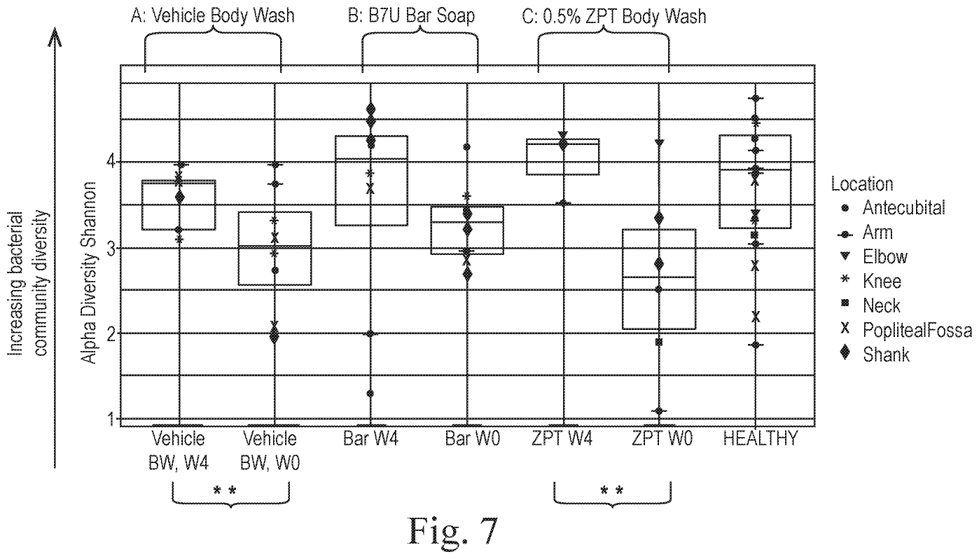

Results, shown in FIG. 7, reveal that skin microbiota diversity from the various skin regions sampled was restored by zinc pyrithione (BCP2ZPT) and, to a lesser extent, by the vehicle wash (BCP2). Thus, the action of compositions according to the disclosure selectively reduce the relative abundance of some bacterial genera, notably Staphylococcus, and increase the relative abundance of other bacterial genera, and these selective effects on skin bacterial genera act to modulate the skin microbiota in a manner that restores a wild-type appearance to the skin microbiota in terms of diversity and relative abundances.

The data presented in Example 4 and elsewhere in the disclosure reveal that compositions comprising a zinc ionophore (e.g., zinc pyrithione), a biocompatible surfactant, and a lipid provide a selective therapeutic useful in reducing the relative abundance of Staphylococcus and modulating the relative abundances of a number of other skin bacterial genera in treating conditions characterized by an aberrant skin microbiota diversity, such as atopic dermatitis, with or without lesions, skin dysbiosis, or acne. In addition, the data establish the value of assessing skin microbiota diversity in identifying a propensity or likelihood to develop the sound, but unhealthy, skin associated with a variety of skin disorders such as atopic dermatitis (with or without lesions), skin dysbiosis, or acne.

### Examples/Combinations

Aspects of the disclosure are also described in the following enumerated paragraphs.

A. A method of selectively increasing microbial diversity of the skin microbiota of amenable skin comprising administering an effective amount of a multi-phase skin improvement composition comprising a cleanser comprising a zinc compound and a lathering biocompatible surfactant, and a benefit agent comprising a lipid, thereby selectively increasing the microbial diversity of the skin microbiota compared to the microbial diversity of the skin microbiota prior to administration of the multi-phase skin improvement composition.

B. The method of paragraph A wherein the zinc compound is zinc monoglycerolate or a zinc ionophore.

C. The method of paragraph A or paragraph B wherein the zinc compound is zinc pyrithione or zinc pyridinethione.

D. The method of any of paragraphs A-C wherein 0.1-2.0 μg zinc pyrithione is administered per cm2 of skin.

E. The method of any of paragraphs A-D wherein the zinc compound is administered as a body wash for at least four weeks.

F. The method of any of paragraphs A-E wherein the biocompatible surfactant is a non-ionic surfactant or an anionic surfactant.

G. The method of paragraph F wherein the anionic surfactant is sodium trideceth(n) sulfate, where n is from 0.5 to 2.7.

H. The method of any of paragraphs A-G wherein the lipid is petrolatum, glyceryl monooleate, glycerin, ceramide, cholesterol, a fatty acid, a triglyceride, a phospholipid, or any combination thereof.

I. The method of any of paragraphs A-H wherein the lipid composition is petrolatum or a mixture of petrolatum and glyceryl monooleate.

J. The method of any of paragraphs A-I wherein 20-200 μg lipid is administered per cm² skin.

K. The method of any of paragraphs A-J wherein the microbial diversity of the skin microbiota is increased relative to the microbial diversity of the skin prior to administration of the multi-phase skin improvement composition, by selectively decreasing the skin level of Staphylococcus relative to its level prior to administration of the multi-phase skin improvement composition.

L. The method of paragraph K wherein the Staphylococcus is Staphylococcus aureus or Staphylococcus epidermidis.

M. The method of any of paragraphs A-L wherein the microbial diversity of the skin microbiota is increased, relative to the microbial diversity of the skin microbiota prior to administration of the multi-phase skin improvement composition, by selectively increasing the level on the skin of at least one of Propionibacterium, Corynebacterium or Streptococcus, relative to its level prior to administration of the multi-phase skin improvement composition.

N. The method of any of paragraphs K-M wherein the change in level leads to an increase in the Shannon index of at least 20%.

Each of the references cited herein is incorporated by reference herein in its entirety, or in relevant passage, as would be apparent from the context of its citation. The disclosed subject matter has been described with reference to various specific examples and techniques. It should be understood, however, that many variations and modifications may be made while remaining within the spirit and scope of the disclosed subject matter.

1. A method of selectively increasing microbial diversity of the skin microbiota of amenable skin comprising selecting a rinse-off multi-phase skin improvement composition comprising a cleansing phase comprising an anti-microbial compound, and a lathering biocompatible surfactant; and a benefit phase comprising a benefit agent comprising a lipid; and administering an effective amount of the multi-phase skin improvement composition, thereby selectively increasing the microbial diversity of the skin microbiota compared to the microbial diversity of the skin microbiota prior to administration of the multi-phase skin improvement composition.

2. The method of claim 15 wherein the zinc compound is zinc monoglycerolate or a zinc ionophore.

3. The method of claim 15 wherein the zinc compound is zinc pyrithione.

4. The method of claim 15 wherein 0.1-2.0 μg of the zinc compound is administered per cm² of skin.

5. The method of claim 4 wherein the zinc compound is administered as a body wash for at least four weeks.

6. The method of claim 1 wherein the biocompatible surfactant is a non-ionic surfactant or an anionic surfactant.

7. The method of claim 6 wherein the anionic surfactant is sodium laureth(n) sulfate, where n is from 1 to 3.

8. The method of claim 1 wherein the lipid is petrolatum, glyceryl monooleate, glycerin, ceramide, cholesterol, a fatty acid, a triglyceride, a phospholipid, or any combination thereof.

9. The method of claim 1 wherein the lipid composition is petrolatum or a mixture of petrolatum and glyceryl monooleate.

10. The method of claim 1 wherein 20-200 μg lipid is administered per cm² skin.

11. The method of claim 1 wherein the microbial diversity of the skin microbiota is increased relative to the microbial diversity of the skin prior to administration of the multi-phase skin improvement composition, by selectively decreasing the skin level of Staphylococcus relative to its level prior to administration of the multi-phase skin improvement composition.

12. The method of claim 11 wherein the Staphylococcus is Staphylococcus aureus or Staphylococcus epidermidis.

13. The method of claim 1 wherein the microbial diversity of the skin microbiota is increased, relative to the microbial diversity of the skin microbiota prior to administration of the multi-phase skin improvement composition, by selectively increasing the level on the skin of at least one of Propionibacterium, Corynebacterium or Streptococcus, relative to its level prior to administration of the multi-phase skin improvement composition.

14. The method of claim 13 wherein the change in level leads to an increase in the Shannon index of at least 20%.

15. The method of claim 1, wherein the anti-microbial compound comprises a zinc-containing compound; carvacrol; helional; menadione; symclairol; alizarin; bardic 2250; chlorhexidine-digluconate; chlorohexenol; 4-isopropyl-3-methylphenol; octopirox, 2-methoxy-1,4-naphthoquinone; 5-hydroxy-1,4-naphthoquinone; 1,2-dodecanediol; phloretin; quercetin hydrate; propyl gallate; methyl 3,4,5-trihydroxybenzoate; octyl gallate; lauryl gallate; ellagic acid tree bark; cinnamic aldehyde; geraniol; thymol; hinokitiol; chrysin; or a combination thereof.

16. A method for selectively increasing the diversity of skin microbiota of a subject with an amenable skin condition, comprising selecting a rinse-off multi-phase skin improvement composition which selectively targets Staphylococcus, wherein the multi-phase skin improvement composition comprises a cleansing phase comprising a surfactant and zinc pyrithione; and a benefit phase comprising a benefit agent.

17. The method of claim 16 wherein 0.1-2.0 μg zinc pyrithione is administered per cm² of skin.
